(19) United States
(12) Patent Application Publication
(10) Pub. No.: US 2014/0018818 A1
(43) Pub. Date: Jan. 16, 2014

(54) SYSTEM AND METHOD OF IMPLANTING A MEDICAL DEVICE

(71) Applicants: Zoltan Somogyi, Simi Valley, CA (US); Edward Karst, South Pasadena, CA (US); Gene A. Bornzin, Simi Valley, CA (US); John W. Poore, South Pasadena, CA (US); Richard Samade, Northridge, CA (US); Didier Theret, Porter Ranch, CA (US)

(72) Inventors: Zoltan Somogyi, Simi Valley, CA (US); Edward Karst, South Pasadena, CA (US); Gene A. Bornzin, Simi Valley, CA (US); John W. Poore, South Pasadena, CA (US); Richard Samade, Northridge, CA (US); Didier Theret, Porter Ranch, CA (US)

(73) Assignee: PACESETTER, INC., Sylmar, CA (US)

(21) Appl. No.: 13/547,791

(22) Filed: Jul. 12, 2012

(51) Int. Cl. A61N 1/05

(52) U.S. Cl. 606/129

(57) ABSTRACT

A system for implanting an implantable medical device (IMD) within a patient may include a main handle assembly having proximal and distal ends, a device-connection control handle connected to the proximal end of the main handle assembly, an introducer connected to the distal end of the main handle assembly, and a connection tool extending from the introducer. The connection tool may include a device-engaging member configured to change at least one of shape or orientation to selectively connect to and disconnect from the IMD. The device-connection control handle may be operatively connected to the device-engaging member and the device-connection control handle may be configured to manipulate the device-engaging member between connected and disconnected states by changing the at least one of the shape or orientation.

### FIELD OF THE INVENTION

Embodiments generally relate to an implantable medical device, and, more particularly, to a system and method of implanting a medical device.

### BACKGROUND

Numerous medical devices exist today, including but not limited to electrocardiographs (“ECGs”), electroencephalographs (“EEGs”), squid magnetometers, implantable pacemakers, implantable cardioverter-defibrillators (“ICDs”), neurostimulators, electrophysiology (“EP”) mapping and radio frequency (“RF”) ablation systems, and the like (hereafter generally “implantable medical devices” or “IMDs”). IMDs commonly employ one or more leads with electrodes that either receive or deliver voltage, current or other electromagnetic pulses (generally “energy”) from or to an organ or tissue (collectively hereafter “tissue”) for diagnostic or therapeutic purposes.

Typically, an IMD is outside of the heart. To sense atrial cardiac signals and to provide right atrial chamber stimulation therapy, the IMD is coupled to an implantable right atrial lead including at least one atrial tip electrode that typically is implanted in the patient's right atrial appendage. The right atrial lead may also include an atrial ring electrode to allow bipolar stimulation or sensing in combination with the atrial tip electrode.

Notably, a substantial portion of the leads, as well as the IMD itself are outside of the patient's heart. Consequently, bacteria and the like may be introduced into the patient's heart through the leads, as well as the IMD, thereby increasing the risk of infection within the heart.

Additionally, because the IMD is outside of the heart, the patient may be susceptible to Twiddler's syndrome, which is a condition caused by the shape and weight of the IMD itself. Twiddler's syndrome is typically characterized by a subconscious, inadvertent, or deliberate rotation of the IMD within a subcutaneous pocket formed in the patient. In one example, a lead may retract and begin to wrap around the IMD. Also, one of the leads may dislodge from the endocardium and cause the IMD to malfunction. Further, in another typical symptom of Twiddler's syndrome, the IMD may stimulate the diaphragm, vagus, or phrenic nerve, pectoral muscles, or brachial plexus. Overall, Twiddler's syndrome may result in sudden cardiac arrest due to conduction disturbances related to the IMD.

Further, locating the IMD outside of the heart may cause discomfort to the patient, erode skin proximate the IMD in its subcutaneous pocket, and the like.

Intra-cardiac IMDs have been developed to alleviate the drawbacks of conventional IMDs. Typically, an intra-cardiac IMD is introduced into the heart through a catheter. However, trans-catheter delivery of an entire IMD within a heart typically requires specialized tools. Often, the specialized tools are complex and may be difficult to manipulate and operate.

### SUMMARY

Certain embodiments provide a system for implanting an implantable medical device (IMD) within a patient. The system may include a main handle assembly having proximal and distal ends, a device-connection control handle connected to the proximal end of the main handle assembly, an introducer connected to the distal end of the main handle assembly, and a connection tool extending from the introducer. The connection tool may include a device-engaging member configured to change at least one of shape or orientation to selectively connect to and disconnect from the IMD. The device-connection control handle is operatively connected to the device-engaging member and the device-connection control handle is configured to manipulate the device-engaging member between connected and disconnected states by changing the at least one of the shape or orientation.

The device-engaging member may include connectors that radially expand and contract outward and inward with respect to a longitudinal axis of the connection tool. Optionally, the device-engaging member may include connectors that move laterally inward and outward to change the shape between the disconnected and connected states.

The connection tool may include a base, opposed spreadable connectors moveably secured to the base, and a stylet configured to move between the opposed spreadable connectors in order to spread the opposed spreadable connectors into the connected state, and recede away from the opposed spreadable connectors so that the opposed spreadable connectors retract toward one another into the disconnected state. The IMD may include either a recessed cavity or a protuberance. The opposed spreadable connectors may be configured to engage the recessed cavity or the protuberance in the connected state. The opposed spreadable connectors may be configured to pivot with respect to the base. The opposed spreadable connectors may be configured to linearly move with respect to the base.

In an embodiment, the connection tool may include a deflectable member. The deflectable member may include an elongated braid having a metal mesh.

The connection tool may include a protective covering member configured to protect the IMD during navigation to an implantation site. The IMD may be moved out of the protective covering member at the implantation site.

In an embodiment, the device-engaging member may include a securing cuff operatively connected to the device-connection control handle, and at least one securing member configured to engage the IMD. The securing cuff may be configured to engage the at least one securing member. The securing cuff may be connected to a coupling member that is operatively connected to the device-connection control handle. Optionally, the securing cuff may be connected to the coupling member through at least one beam. In an embodiment, the securing cuff and the coupling member may magnetically attract or repel one another.

In an embodiment, one of the device-engaging member or the IMD may include a distal block configured to pass into a securing channel formed in the other of the device-engaging member or the IMD.

In an embodiment, the device-engaging member may include one of a securing feature or a protuberance, while the IMD may include the other of the securing feature or the protuberance. The securing feature receives and retains the protuberance when the IMD is pulled into the device-engaging member.

In an embodiment, the device-engaging member may include securing members pivotally connected to one another. The device-engaging member may be configured to be pushed out of and pulled into a deflectable member through operation of the device-connection control handle.

In an embodiment, one of the device-engaging member or the IMD may include a key, and the other of the device-engaging member or the IMD may include a channel configured to receive the key. The channel may include an insertion portion connected to a retaining portion. The device-engaging member may be maneuvered into the retaining portion by the device-connection control handle in the connected orientation.

Certain embodiments provide a method of connecting an implantable medical device (IMD) to a device-engaging member extending from a connection tool of an implantation system. The method may include aligning the device-engaging member with a connecting interface of the IMD, moving the device-engaging member into or onto the connecting interface, manipulating a device-connection control handle of the implantation system, changing at least one of shape or orientation of the device-engaging member, by the manipulating, from a disconnected state to a connected state while the device-engaging member is on or within the connecting interface, and connecting the IMD to the device-engaging member through the changing at least one of shape or orientation.

The changing at least one of shape or orientation may include radially expanding or contracting connectors of the device-engaging member outward or inward with respect to a longitudinal axis of the connection tool. Optionally, the changing at least one of shape or orientation may include laterally moving connectors of the device device-engaging member inward or outward.

The manipulating may include rotating the device-connection control handle. Alternatively, the manipulating may include pushing or pulling the device-connection control handle.

Certain embodiments provide a method of implanting an implantable medical device (IMD) within a patient. The method may include connecting the IMD to a device-engaging member at a distal end of a connection tool of an implantation system, maneuvering the IMD to an implantation site through the implantation system, advancing the IMD out of a protective covering member of the connection sheath into the implantation site, manipulating the implantation system to securely anchor the IMD into tissue at the implantation site, manipulating a device-connection control handle of the implantation system, changing at least one of shape or orientation of the device-engaging member by the manipulating the device-connection control handle, and disconnecting the device-engaging member from the IMD through the changing at least one of shape or orientation of the device-engaging member.

The changing at least one of shape or orientation may include radially expanding or contracting connectors of the device-engaging member outward or inward with respect to a longitudinal axis of the connection tool. Optionally, the changing at least one of shape or orientation may include laterally moving connectors of the device device-engaging member inward or outward.

The manipulating the device-connection control handle may include rotating the device-connection control handle. Alternatively, the manipulating the device-connection control handle may include pushing or pulling the device-connection control handle.

### BRIEF DESCRIPTION OF THE DRAWINGS

FIG. 1 illustrates an isometric view of an implantation system, according to an embodiment.

FIG. 2 illustrates a lateral view of an implantable medical device proximate a connection tool extending from an introducer, according to an embodiment.

FIG. 3 illustrates a transverse cross-sectional view of an introducer, according to an embodiment.

FIG. 4 illustrates an axial cross-sectional view of an introducer through line 4-4 of FIG. 3, according to an embodiment.

FIG. 5 illustrates a lateral view of a device-engaging member and a stylet, according to an embodiment.

FIG. 6 illustrates a lateral view of a connection tool, according to an embodiment.

FIG. 7a illustrates an internal cross-sectional view of a device-engaging member aligned with a recessed cavity of an implantable medical device, according to an embodiment.

FIG. 7b illustrates an internal cross-sectional view of a device-engaging member aligned with a recessed cavity of an implantable medical device, according to an embodiment. FIG. 8a illustrates an internal cross-sectional view of a device-engaging member secured to an implantable medical device, according to an embodiment.

FIG. 8b illustrates an internal cross-sectional view of a device-engaging member secured to an implantable medical device, according to an embodiment

FIG. 9 illustrates an internal cross-sectional view of an IMD, according to an embodiment.

FIG. 10 illustrates a device-engaging member having radially expanded connectors, according to an embodiment.

FIG. 11a illustrates a device-engaging member aligned with a securing protuberance extending outwardly from an implantable medical device, according to an embodiment.

FIG. 11b illustrates an axial cross-sectional view of a securing cuff coupled to a wire, according to an embodiment.

FIG. 11c illustrates an axial cross-sectional view of a securing cuff coupled to a wire, according to an embodiment.

FIG. 12 illustrates a device-engaging member aligned with a securing channel formed through a proximal end of an implantable medical device, according to an embodiment.

FIG. 13 illustrates an axial view of a device-engaging member secured to an implantable medical device, according to an embodiment

FIG. 14 illustrates a lateral view of a device-engaging member of a connection tool and an implantable medical device, according to an embodiment.

FIG. 15 illustrates a connection tool, according to an embodiment.

FIG. 16 illustrates a device-securing member aligned with a securing member extending from an IMD, according to an embodiment.

FIG. 17 illustrates an implantable medical device being implanted within a heart of a patient, according to an embodiment.

FIG. 18 illustrates a flow chart of a method of securing an implantable medical device to a device-engaging member.

FIG. 19 illustrates a flow chart of a method of implanting an IMD into a tissue within a patient, according to an embodiment.

### DETAILED DESCRIPTION

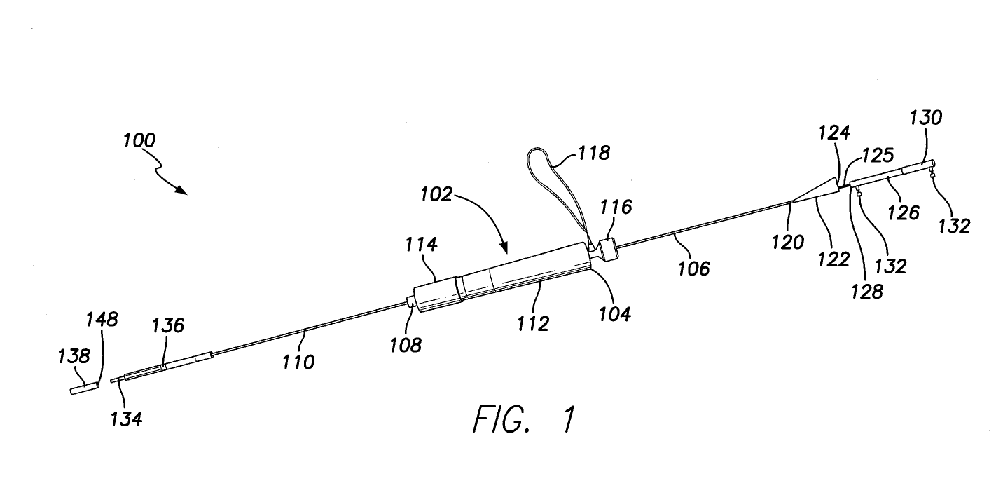

FIG. 1 illustrates an isometric view of an implantation system 100, according to an embodiment. The system 100 includes a main handle assembly 102 having a proximal end 104 connected to a flexible tube 106, such as a catheter, and a distal end 108 connected to an introducer 110, such as a catheter.

The main handle assembly 102 may be formed of metal or plastic and includes a tubular fixed portion 112 operatively connected to a rotatable portion 114. The rotatable portion 114 is configured to be rotated with respect to the fixed portion 112 about a longitudinal axis of the main handle assembly 102. The main handle assembly 102 is sized and shaped to be grasped and manipulated by an operator, such as a physician. As such, the main handle assembly 102 may be ergonomically designed for comfort and operability. The main handle assembly 102 may optionally be various other shapes and sizes. While the main handle assembly 102 is shown as generally tubular or cylindrical, the main handle assembly 102 may be block shaped, for example.

The flexible tube 106 connects to the fixed portion 112 of the main handle assembly 102. The flexible tube 106 defines an internal passage (not shown). The flexible tube 106 connects to a central channel (not shown) formed through the main handle assembly 102.

As shown, a connecting nut 116, such as a Touhy-borst adapter, may secure to the fixed portion 112 of the main handle assembly 102 at the proximal end 104. The connecting nut 116 may secure a drip line 118 to the central channel of the main handle assembly 102. The drip line 118 may be used to introduce a flushing fluid, such as water, into the central channel 104 and the introducer 110, which connects to the central channel, in order to flush or otherwise remove fluid, such as blood, from the system 100. Optionally, the drip line 118 may be connected to a vacuum that suctions fluid out of the system 100 through the drip line 118.

The flexible tube 106 has a proximal end 120 that connects to a rigid support 122, such as a funnel, bracket, rigid end terminal, or the like. The rigid support 122 may be a funnel having an open end 124 that connects to the internal passage of the flexible tube 106. The rigid support 122 is configured to retain a wire 125 therein that passes through the system 100. An operator may operate a device-engaging member 134 by way of a graspable device-connection control handle 126 connected to a proximal end 128 of the wire 125. While the rigid support 122 is shown and described as a funnel, the rigid support 122 may be various other shapes and sizes. For example, the rigid support 122 may be sized and shaped similar to the main handle assembly 102.

The device-connection control handle 126 may be formed of metal or plastic and may include a main body 130 having one or more graspable protuberances 132 that extend outwardly from the main body 130. The protuberances 132 may be normal to the longitudinal axis of the main body 130. The protuberances 132 may be screws, bolts, or other such fasteners that thread through reciprocal through-holes formed in the main body. Distal ends (not shown) of the protuberances 132 may securely engage the wire 125 within the main body 130 so that rotation of the graspable device-connection control handle 126 produces a common rotation in the wire 125. Alternatively, the protuberances 132 may be various other protruding, tactile features, such as posts, tabs, or the like. Also, alternatively, the graspable device-connection control handle 126 may not include the protuberances 132. Instead, the wire 125 may be securely fixed within the main body 130, such as through adhesives, bonding, or the like. Moreover, the device-connection control handle 126 may be any type of handle configured to be grasped and manipulated by an operator. For example, the device-connection control handle 126 may be or include a tubular plastic cap secured to the end of the wire 125. The tubular plastic cap may include tactile features, such as ribs, embossments, or the like, configured to provide a better grip for an operator.

The device-connection control handle 126 is used to operate and manipulate a device-engaging member 134 located at a distal end 136 of the introducer 110. (e.g., to rotate, translate, bend, deflect and otherwise move the device-engaging member 134) The device-engaging member 134 connects to the device-connection control handle 126 through the wire 125, which passes through the flexible tube 106, into the main handle assembly 102, and through the introducer 110. The device-engaging member 134 is configured to removably secure to an implantable medical device (IMD) 138. That is, the device-engaging member 134 is configured to selectively connect to and disconnect from the IMD 138.

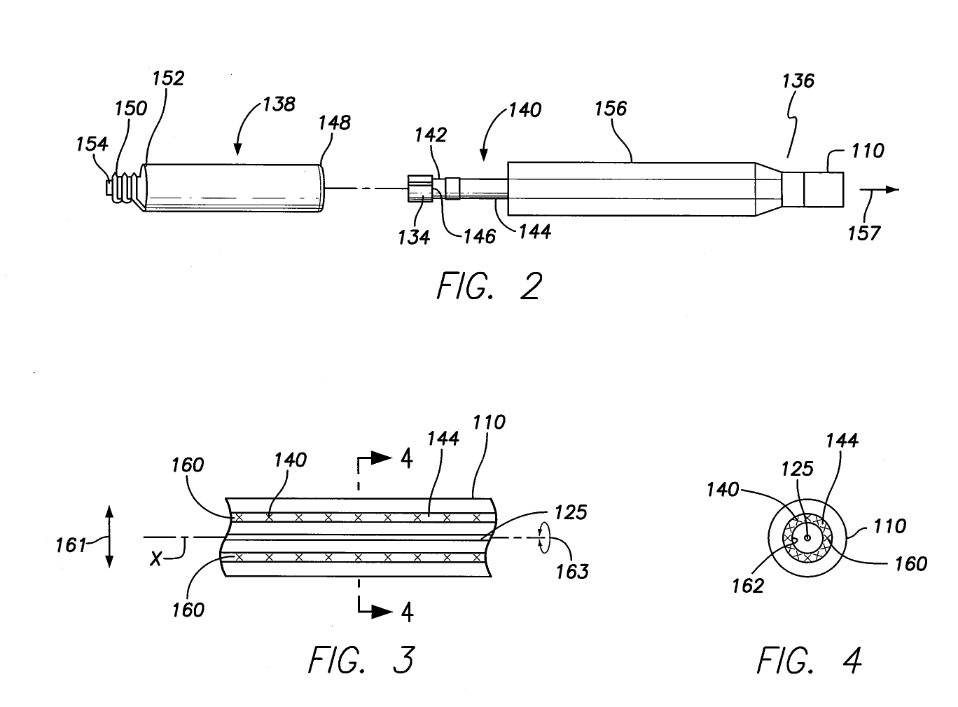

FIG. 2 illustrates a lateral view of the IMD 138 proximate a connection tool 140 extending from the introducer 110, according to an embodiment. The connection tool 140 may be a device configured to deflect, advance through, retract into, rotate, and the like with respect to the introducer 110. The connection tool 140 may include one or more of a flexible, deflectable rotatable pusher, retractor, extender, and the like. The connection tool 140 is elongated along a longitudinal axis that extends generally in the direction of arrow 157.

The connection tool 140 includes the device-engaging member 134 secured to a distal end 142 of a wire-reinforced plastic deflectable member 144, such as a lumen, catheter, tube, or the like. The deflectable member 144 may bend along a slow or sharp curve, in response to user controls at the control handle 126, in a flared or deflection direction with respect to the length of the connection tool 140. Within the introducer 110, the wire 125 (shown in FIG. 1) may be retained within a central channel defined by the deflectable member 144. A distal end of the wire 125 secures to a base 146 of the device-engaging member 134, which is fixed to the distal end 142 of the deflectable member 144. For example, the base 146 may fit within the central channel of the deflectable member 144 and be adhesively bonded thereto.

The wire 125 may also be coaxially securely retained within the deflectable member 144, such as through adhesives. Thus, rotation of the wire 125 may cause a common rotation of the deflectable member 144. Optionally, the wire 125 may not be adhesively secured to the deflectable member 144. In this embodiment, because the base 146 of the device-engaging member 134 is securely fixed to the deflectable member 144, rotation of the wire 125 is translated to the device-engaging member 134, which, in turn, causes rotation of the deflectable member 144.

The device-engaging member 134 is configured to secure to a proximal end 148 of the IMD 138 in order to deliver, fixate and remove the IMD 138. The device engaging member 134 is configured to change shape, such as by laterally expanding and contracting, toward and away from the longitudinal axis of the connection tool 140. The device-engaging member 134 may move between expanded and collapsed states. As one example, the device-engaging member 134 may expand in a radial direction outward from an axis defined by the wire 125 to an expanded state, to become affixed to a counterbore portion in the IDM 138. The IMD 138 may be one of various types of implantable devices, such as, for example, an implantable pacemaker, a cardiac resynchronization therapy (CRT) device, an implantable cardioverter-defibrillator (“ICD”), neurostimulator, or the like. The IMD 138 may be configured for DDDR pacing (atrial and ventricular pacing, atrial and ventricular sensing, dual response and rate-adaptive, used for dual chamber pacemakers). A securing helix 150 extends from a distal end 152 of the IMD 138. The securing helix 150 may be a coiled helical wire having a sharp distal tip 154 that is configured to anchor the IMD 138 into internal tissue wall of a patient.

When connected to the device-engaging member 134, the IMD 148 may be retracted within a protective covering member 156, such as a sleeve, sheath, shroud, collar, or the like, that extends from the distal end 136 of the introducer 110. The protective covering member 156 may be formed of a flexible plastic that protects the IMD from snagging patient anatomy. The deflectable member 144 may be pulled back in the direction of arrow 157, thereby pulling back the IMD 148 within the protective covering member 156. The protective covering member 156 may surround the helix 150 so that the helix 150 does not snag, cut, or otherwise damage internal tissue as the IMD 138 is navigated toward an implantation site. Once the IMD 138 reaches the implantation site, the deflectable member 144 may be pushed out of the protective covering member 156 so that the helix 150 may engage tissue at the implantation site.

The device-engaging member 134 may include connectors that may radially expand and contract outward and inward with respect to a longitudinal axis of the connection tool. The device-engaging member 134 may include connectors that move laterally inward and outward to change the shape between disconnected and connected states.

FIG. 3 illustrates a transverse cross-sectional view of the introducer 110, according to an embodiment. The deflectable member 144 may be formed of an elongated braid 160, which may be made of steel or wire mesh, and/or have a honeycomb pattern that resists compression or extension along the length of the deflectable member 144. The braid 160 is flexible in directions 161 that are perpendicular to the longitudinal axis X of the connection tool 140 in order to be bent side-to-side during implant and following implant. The mesh or honeycomb configuration of the braid 160 affords strong resistance to torque about the length of the connection tool 140 when turned in the rotational direction 163 about the longitudinal axis X. It is desirable to be resistant to torque in order that, during implant, when a rotational force is applied to one end of the connection tool 140, substantially all of such rotational force is conveyed along the length of the connection tool 140 to the opposite end. The braid 160 facilitates delivery of rotational forces and longitudinal pressure to the connection tool 140 during implant.

FIG. 4 illustrates an axial cross-sectional cross-sectional view of the introducer 110 through line 4-4 of FIG. 3, according to an embodiment. As shown, the wire 125 may be coaxial with the introducer 110 and the connection tool 140. As noted above, the wire 140 may be adhesively secured to interior walls 162 of the deflectable member 144. Optionally, the distal end of the wire 125 may be merely fixed to the base 146 of the device-engaging member 134, while the length of the wire 125 within the deflectable member 144 is unsecured within the deflectable member 144.

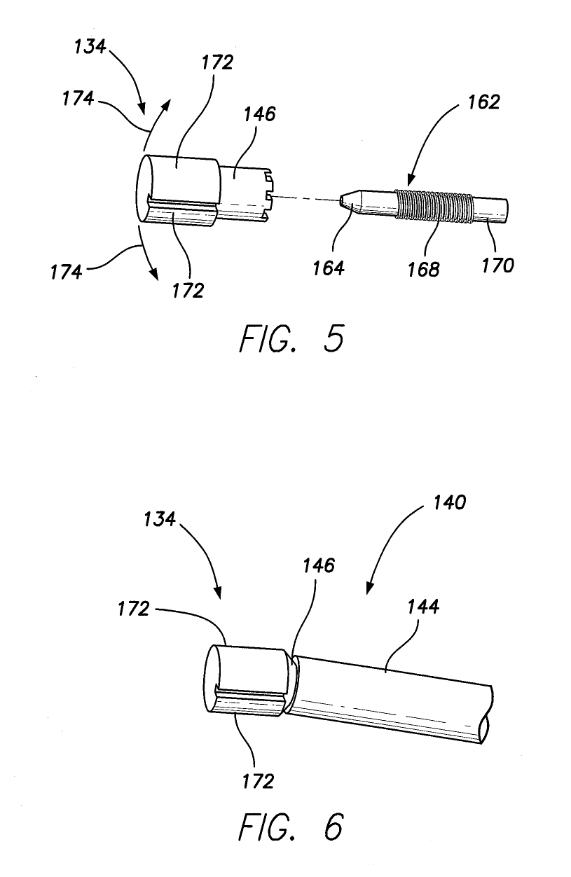

FIG. 5 illustrates a lateral view of the device-engaging member 134 and a stylet 162, according to an embodiment. Both the device-engaging member 134 and the stylet 162 may be formed of metal, such as Titanium or stainless steel, or plastic. The stylet 162 may be a rigid connecting member that is connected to, or forms, the distal end of the wire 125. The stylet 162 may include a distal, beveled lead-in tip 164 integrally connected to a cylindrical shaft 166 having an intermediate threaded portion 168, which, in turn, connects to a proximal end 170. The proximal end 170 may be integrally connected to a distal end of the wire 125 (shown in FIG. 1), so that rotation of the wire 125 causes a common rotation of the stylet 162. Optionally, the proximal end 170 may be separate and distinct from the wire 125, and may be secured thereto, through welding, bonding, or the like.

The device-engaging member 134 includes the fixed base 146 operatively connected to connectors 172, such as expandable, spreadable jaws, arms, legs, or the like. The connectors 172 cooperate with one another to form a securing collet, flange, or the like. The stylet 162 is configured to spread the connectors 172 open in lateral directions of arrows 174, thereby changing the shape of the device-engaging member 134 to a radially expanded connected state. The stylet 162 is also configured to collapse the connectors 172 against one another in a closed or radially contracted disconnected state.

The lead-in tip 164 is received and retained by an internal, threaded passage (not shown) formed through the base. The intermediate threaded portion 168 of the stylet 162 threadably engages the internal passage to secure the stylet 162 to the device-engaging member 134. In order to expand the connectors 172, the stylet 170 is rotated so that the lead-in tip 164 advances between the connectors 172. With continued urging, the stylet 162 spreads the connectors 172 apart in the lateral directions of arrows 174 to the expanded state. As explained below, when the connectors 172 are spread apart within a recessed cavity or counterbore formed in a proximal end of the IMD 138, the IMD 138 is securely attached to the device-engaging member 134. In order to detach the device-engaging member 134 from the IMD 138, the stylet 162 is rotated in an opposite direction so that the lead-in tip 164 retreats toward the base 146 of the device-engaging member 134. As the lead-in tip 164 disengages the connectors 172, the spring tension of the connectors 172 causes them to retract in directions opposite from 174, to the radially contracted disconnected state and the connectors 172 disengage the IMD 138. When in the contracted disconnected state, the connectors 172 collectively define an outer envelope that is smaller than an interior envelope defined by the recessed cavity in the IDM 138. When in the expanded connected state, the connectors 172 collectively define an outer envelope that frictionally engages, and is a common size as the interior envelope of the recessed cavity in the IDM 138.

FIG. 6 illustrates a lateral view of the connection tool 140, according to an embodiment. As shown in FIG. 5, the deflectable member 144 surrounds the wire 125 (hidden from view in FIG. 6) and the stylet 162 (hidden from view in FIG. 6). The deflectable member 144 may be adhesively secured, bonded, or the like, to the base 146 of the deflectable member 134.

Referring to FIGS. 1-6, in order to connect the IMD 138 to the device-engaging member 134, a recessed cavity formed in the proximal end 148 of the IMD 138 is aligned with the distal lead-in tip 164 of the device-engaging member 134. Examples of recessed cavities are discussed below. The lead-in tip 164 is then inserted into the recessed cavity. An operator then rotates the graspable device-connection control handle 126 of the system 100 in a connecting or securing direction. That is, the operator manipulates the device-connection control handle 126 with a single, simple movement, such as a rotation in a first securing or connecting direction. Rotation of the graspable device-connection control handle 126 causes a common rotation in the wire 125, which passes through the flexible tube 106, the main handle assembly 102, and the introducer 110. Because the distal end of the wire 125 is secured to the stylet 162, which is threadably secured within the base 146 of the device-engaging member 134, rotation of the wire 125 causes the stylet 162 to threadably advance between the connectors 172 of the device-engaging member 134. As the connectors 172 spread apart within the recessed cavity of the IMD 136, the envelope defined by the combined diameter of the spread, expanded connectors 172 is greater than the outlet of the recessed cavity. As such, the IMD 138 is secured to the device-engaging member 134.

In order to release the IMD 138 from the device-engaging member 134, the graspable device-connection control handle 126 is rotated in a disconnecting direction that is opposite from the connecting or securing direction. As such, the stylet 162 retreats from the connectors 172. As the stylet 162 retreats back toward the base 146 of the device-engaging member 134, the spring tension of the connectors 172 causes them to move back toward one another, such that the envelope defined by their combined diameter is equal to or less than the outlet of the recessed cavity form in the proximal end 148 of the IMD 138. As such, the device-engaging member 134 may disconnect from the IMD 138.

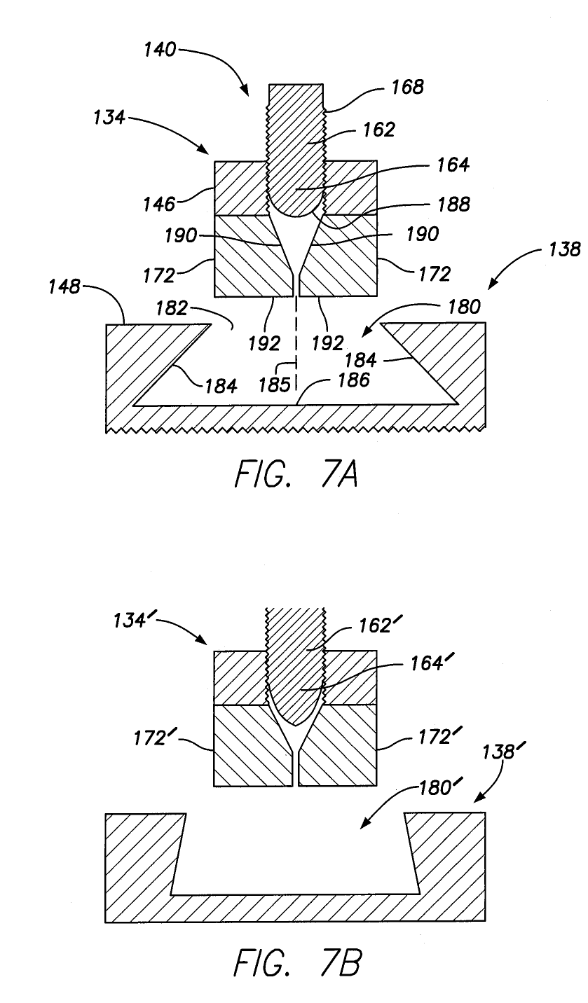

FIG. 7a illustrates an internal cross-sectional view of the device-engaging member 134 aligned with a connecting interface, such as a recessed cavity 180, of the IMD 138, according to an embodiment. The recessed cavity 180 may be a counterbore and is defined by an inlet 182 that connects to angled outer walls 184 that connect to a base 186. The angled outer walls 184 angle away from a longitudinal axis 185 of the IMD 138 toward the base 186. The diameter of the recessed cavity 180 expands from the inlet 182 toward the base 186. As such, the inlet 182 has a smaller diameter than the base 186. The recessed cavity 180 may be a frusto-conical shape, although various other shapes having an inlet smaller than a base may be used.

As shown in FIG. 7a, the stylet 162 is threadably engaged within an internal passage 188 of the base 146. However, in a disconnected state, the lead-in tip 164 is not positioned between the connectors 172. The connectors 172 have interior walls 190 that angle inwardly toward distal ends 192. Thus, the distance between the interior walls 190 of the opposed connectors 172 proximate the base 146 is greater than the distance between the interior walls 190 of the opposed connectors 172 proximate the distal ends 192.

In order to connect the device-engaging member 134 to the IMD 138, the connection tool 140 is urged into the recessed cavity 180. Once inside the recessed cavity 180, the stylet 162 is advanced between the connectors 172.

The interior of the recessed cavity 180 and the exterior of the connectors 172 may have frictional features, such as embossments, ridges, ribs, or other such protrusions that are configured to frictionally engage one another when the connectors 172 are expanded in a connected state within the recessed cavity. The frictional features increase the strength of the connecting interface between the expanded connectors 172 and the recessed cavity 172.

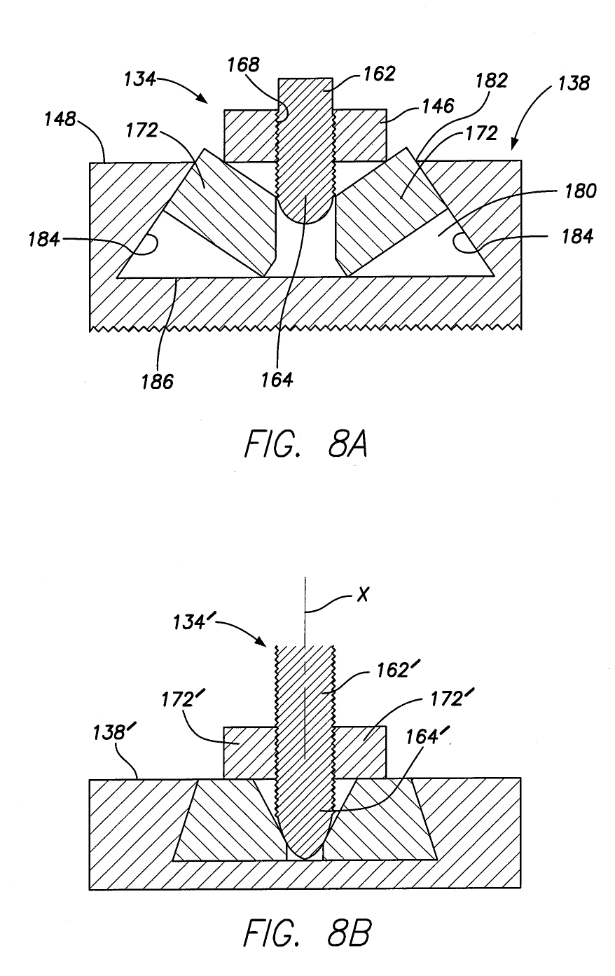

FIG. 8a illustrates an internal cross-sectional view of the device-engaging member 134 secured to the IMD 138, according to an embodiment. As noted above, the stylet 162 is rotated in a securing or connecting direction so that the lead-in tip 164 moves between the connectors 172. As the lead-in tip 164 moves further between the connectors 172, the tapered walls 190 abut against the lead-in tip 164. With increased movement in the securing or connecting direction, the lead-in tip 164 spreads the connectors 172, which may be pivotally connected to the base 146, apart. That is, the distance between the opposed interior walls 190 of the opposed connectors 172 as the lead-in tip 164 continues to advance is smaller than the diameter of the lead-in tip 164. As such, the lead-in tip 164 forces the connectors 172 apart.

When in the connected state, the spread connectors 172 have an envelope with a diameter that exceeds that of the inlet 182 of the recessed cavity 180. Therefore, the spread connectors 172 are unable to retreat back through the inlet 182. Consequently, the device-engaging member 134 is secured to the IMD 138.

In order to remove the device-engaging member 134 from the IMD 138, the stylet 162 is rotated in a direction opposite to that of the securing direction. As the stylet 162 retreats back into the base 146, the stylet 162 disengages the connectors 172. The spring tension of the connectors 172 causes the connectors 172 to move back toward one another. Once the connectors 172 are at their at-rest positions (as shown in FIG. 7), the combined diameter of the connectors 172 is less than inlet 182 of the recessed cavity 182. Therefore, the device-engaging member 134 may then be removed from the IMD 138.

While the stylet 162 is described as having an intermediate threaded portion 168 connected to a smooth, beveled lead-in tip 164, the entirety of the stylet 162 may alternatively be fully-threaded. Moreover, the stylet 162 may have a uniform diameter throughout, instead of having the beveled lead-in tip 164.

Alternatively, instead of the frusto-conical shape of the recessed cavity 180 shown in FIGS. 7a and 8a, the recessed cavity 180 may simply be a cylindrical, tubular shape having straight side walls. When the connectors 172 are spread, the connectors 172 may merely exert an engaging force into the side walls that secures the device-engaging member 134 to the IMD through an interference fit, for example.

Additionally, alternatively, the IMD 138 may include a connection stud or other such protuberance extending from the proximal end 148, instead of having a recessed cavity 180 formed therein. In this embodiment, the stylet 162 is advanced between the connectors 172 in the disconnected state so that the connectors 172 are spread apart. In order to connect the device-engaging member 134 to the IMD 138, the wire 125 connected to the stylet 162 is rotated or pulled back so that the stylet 162 retreats from the connectors 172. The natural tension of the connectors 172 may force them together so that they may cooperate to securely grasp onto the connection stud of the IMD 138. Optionally, the connectors 172 may be spring-biased to clamp together when the stylet 162 is removed from between the connectors 172. Additionally, alternatively, the connectors may be magnetically coupled to one another, and/or magnetically attracted to the connection stud to provide a securing, clamping force. Therefore, the disconnected state of the connectors 172 may be expanded, while the connected state of the connectors 172 may be retracted or otherwise closed together.

FIG. 7b illustrates an internal cross-sectional view of a device-engaging member 134′ aligned with a connecting interface, such as a recessed cavity 180′, of an IMD 138′, according to an embodiment. The device-engaging member 134′ is similar to the device-engaging member 134 shown in FIG. 7a, except that the lead-in tip 164′ of the stylet 162′ has a sharper point, and is configured to engage the connectors 172′, which may be spreadable collets, as opposed to the connectors 172, which tend to pivot, as shown in FIG. 8a.

FIG. 8b illustrates an internal cross-sectional view of the device-engaging member 134′ secured to the IMD 138′, according to an embodiment. As shown in FIG. 8b, the connectors 172′ are forced apart by the stylet 162′ and spread open in directions that are perpendicular to the longitudinal axis X of the stylet 162′. The connectors 172′ are wedged into internal walls of the IMD 138′, and are prevented from moving back toward one another by the lead-in tip 164′ and the stylet 162′. In this manner, the connectors 172′ provide an adjustable collet that is able to couple to the IMD 138′.

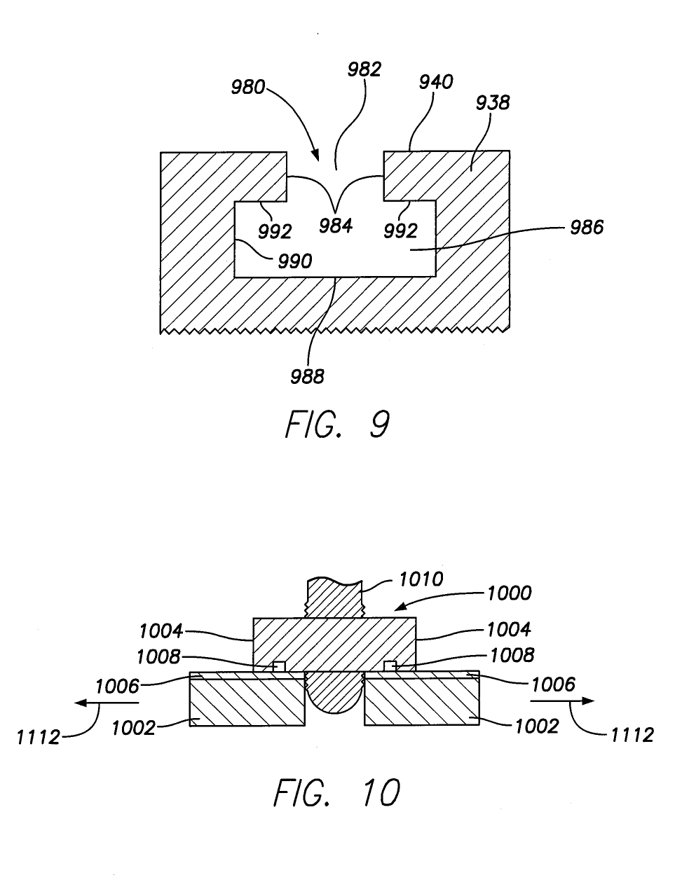

FIG. 9 illustrates an internal cross-sectional view of a proximal end 940 of an IMD 938, according to an embodiment. In this embodiment, the proximal end 940 of the IMD 938 has a connecting interface having a recessed cavity 980 formed therein. The recessed cavity 980 includes an inlet 982 defined by vertical side walls 984. The inlet 982 connects to an expanded chamber 986 defined by a base 988, vertical side walls 990, which, in turn, connect to horizontal beams 992. The recessed cavity 980 is configured to receive the device-engaging member 134 (shown in FIGS. 7 and 8). Optionally, the recessed cavity 980 may be configured to receive and retain a device-engaging member having connectors that expand radially outwardly to form a T-shape.

FIG. 10 illustrates a device-engaging member 1000 having radially expanded connectors 1002, according to an embodiment. The connectors 1002 may be slidably secured to a base 1004 through tracks 1006 that slidably retain protuberances 1008, such as tabs, barbs, posts, or the like, extending from the base 1004. The protuberances 1008 are configured to slide through the tracks 1006. Optionally, the base 1004 may include a track, while the connectors 1002 include protuberances.

As a stylet 1010 advances between the connectors 1002, the connectors 1002 slide apart from one another in the directions of arrows 1112. As such, the expanded end of the device-engaging member 1000 may resemble a T. The device-engaging member 1000 may securely mate with the IMD 938 shown in FIG. 10. When the stylet 1010 retreats from the connectors 1002, the spring tension in the connectors 1002 forces them back toward one another.

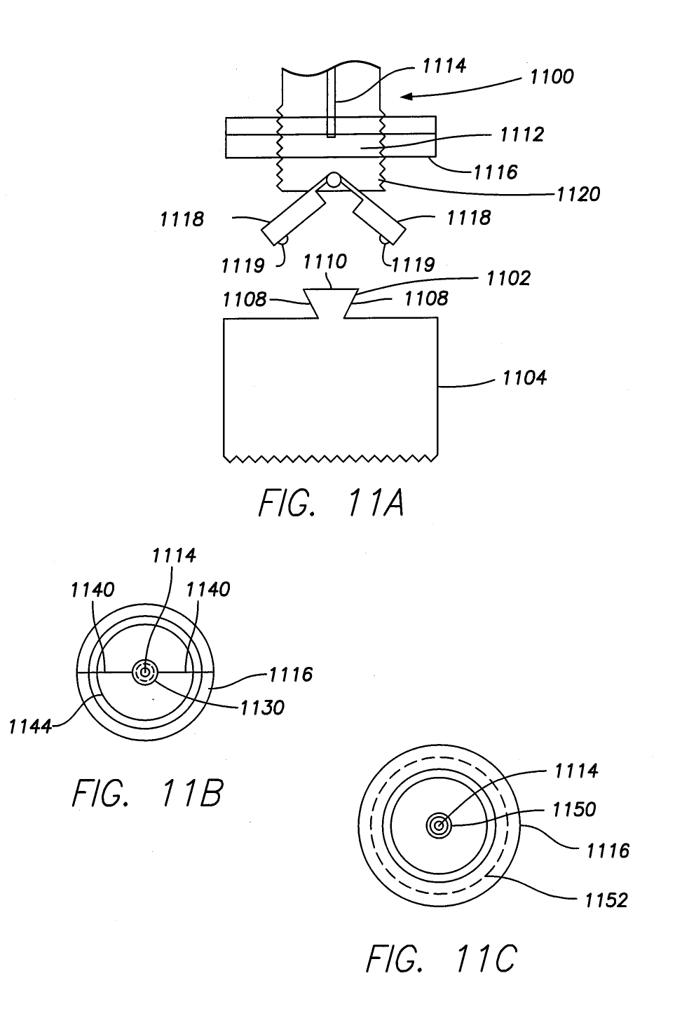

FIG. 11a illustrates a device-engaging member 1100 aligned with a securing protuberance 1102 extending outwardly from an IMD 1104, according to an embodiment. In this embodiment, instead of having a recessed cavity, the IMD 1104 may include a connecting interface having a protuberance 1102, such as a tab, post, barb, stud, or the like, extending therefrom. The protuberance 1102 may be centered about a longitudinal axis of the IMD 1104. The protuberance 1102 is shown having a base 1108 connected to upwardly angled walls 1108 which, in turn, connect to an expanded terminal end 1110. Optionally, however, the protuberance 1102 may simply be a cylindrical post, for example.

The device-engaging member 1100 includes a threaded stylet 1112. A wire 1114, such as the wire 125 shown in FIG. 1, may be operatively coupled to a securing cuff 1116, which threadably secures to or around the stylet 1112. Securing members 1118, such as opposed fingers, an expandable ring, or the like, extends from a distal end 1120 of the stylet 1112.

In order to secure the device-engaging member 1100 to the IMD 1104, the protuberance 1102 is positioned between or within the securing members 1118. The securing cuff 1116 is then urged in a securing or connecting direction toward the securing members 1118. As the securing cuff 1116 engages around portions of the securing members 1118, the securing cuff 1116 causes the securing members 1118 to pivot toward one another. With increased movement of the securing cuff 1116 in the securing direction, the securing members 1118 abut into the protuberance 1102, thereby securing the device-engaging member 1100 in a connected state to the IMD 1104. The securing members 1118 may include gripping members 1119, such as rubber pads, configured to grip the protuberance 1102. Optionally, the securing members 1118 may be include magnets that are magnetically attracted to the protuberance for increased securing force. In order to disconnect the IMD 1104 from the device-engaging member 1100, the securing cuff 1116 is moved in an opposite direction. As the securing cuff 1116 disengages the securing members 1118, the securing members 1118 pivot away from one another to reach a disconnected state and lose contact with the protuberance 1102.

FIG. 11b illustrates an axial cross-sectional view of the securing cuff 1116 coupled to the wire 1114, according to an embodiment. An internal coupling member 1130, such as a collar, flange, or the like, may surround the wire 1114. The coupling member 1130 is connected to the securing cuff 1116 through beams 1140 that pass through channels formed through a deflectable member 1144. The coupling member 1130 may be threadably connected to a threadable portion of a stylet connected to the wire. Thus, rotation of the wire 1114 causes the coupling member 1130, which is secured to the securing cuff 1116, to move in relation to the stylet. In this manner, the securing cuff 1116 may be moved relative to the stylet and wire 1114 over the deflectable member 1144. Optionally, the coupling member 1130 may simply be securely fixed to the wire 1114 or stylet, without any threadable connection. In this embodiment, the wire 1114 is simply pushed or pulled to move the securing cuff 1116 over the deflectable member 1144.

FIG. 11c illustrates an axial cross-sectional view of the securing cuff 1116 coupled to the wire 1114, according to an embodiment. In this embodiment, a coupling member 1130 may be formed of a magnetic material, or include magnets embedded therein. The securing cuff 1116 may include an embedded material 1152, such as another magnetic material or metal, that is attracted to (or repelled from) the magnetic material of or within the coupling member 1130. Thus, as the coupling member 1130 moves, the securing cuff 1116 may move in response thereto, whether in conformity with the movement of the coupling member 1130 (in which the materials are attracted to one another), or opposite to the movement of the coupling member 1130 (in which the materials repel one another).

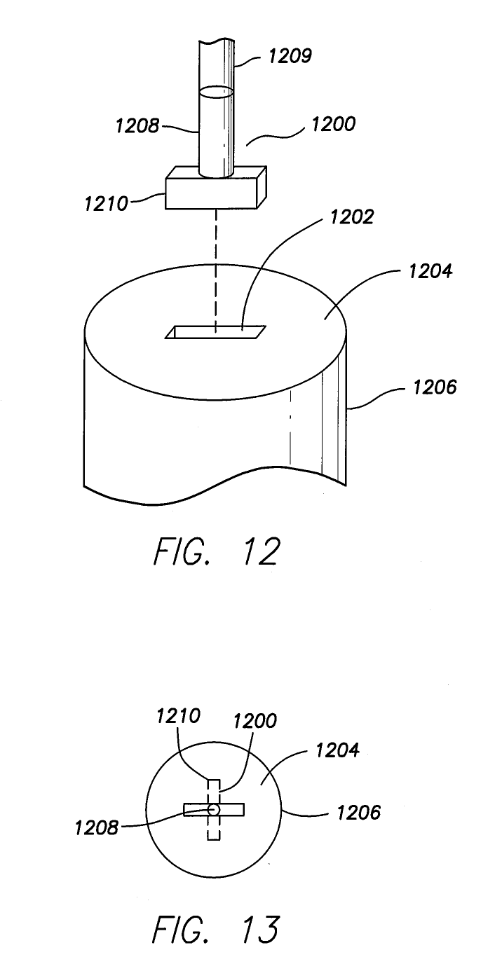

FIG. 12 illustrates a device-engaging member 1200 aligned with a securing channel 1202 formed through a proximal end 1204 of an IMD 1206, according to an embodiment. In this embodiment, the device-engaging member 1200 may include a cylindrical beam 1208 that is directly secured to a wire 1209 that may be operatively connected to the graspable device-connection control handle 126 (shown in FIG. 1). The cylindrical beam 128 integrally connects to a distal block 1210. Thus, rotation of the wire 1209 causes a common rotation in the beam 1208 and the block 1210.

The securing channel 1202 formed in the proximal end 1204 of the IMD 1206 may be shaped and sized to allow the distal block 1210 to pass therein when the distal block 1210 is aligned with the securing channel 1202. The securing channel 1202 opens to an interior chamber within the IMD 1206.

In order to connect the device-engaging member 1200 to the IMD 1206, the distal block 1210 is first aligned with the securing channel 1202 so that it may pass therein. After the distal block 1210 passes through the securing channel 1202, the device-engaging member 1200 is rotated 90 degrees to a connected state so that the block 1210 is unable to retreat back through the securing channel 1202.

FIG. 13 illustrates an axial view of the device-engaging member 1200 in the connected state secured to the IMD 1206, according to an embodiment. As shown, the block 1210 is within the interior chamber of the IMD 1206 and is rotated 90 degrees out of plane with respect to the securing channel 1202. Accordingly, the block 1210 cannot retreat back through the securing channel 1202.

In order to disconnect the IMD 1206 from the device-engaging member 1200, the block 1210 is rotated back 90 degrees so that it is coplanar with the securing channel 1202 in a disconnected state. The block 1210 may then be removed from the IMD 1206.

While the device-engaging member 1200 is described as having a distal block 1210 and the IMD 1206 a reciprocal securing channel 1202, the distal block 1210 may be a securing member formed of various other shapes and sizes. For example, instead of a block, the securing member may be a shaped as a triangle, octagon, or the like, while the securing channel 1202 may be a reciprocal shape. The securing member is rotated out of alignment with the securing channel while within the interior chamber of the IMD 1206 in order to secure the device-engaging member 1200 to the IMD 1206.

Also, alternatively, the IMD 1206 may include a securing member, such as a block, outwardly therefrom, while the device-engaging member 1200 includes a securing channel connected to an interior chamber.

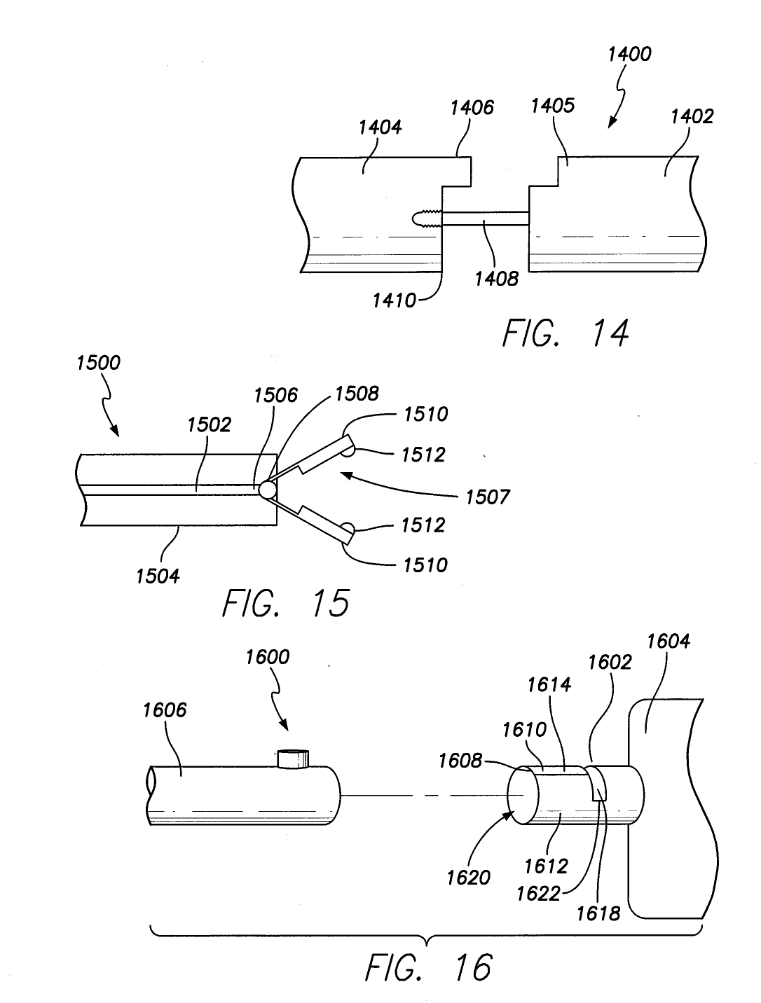

FIG. 14 illustrates a lateral view of a device-engaging member 1400 of a connection tool 1402 and an IMD 1404, according to an embodiment. In this embodiment, the connection tool 1402 may include a securing feature 1405, such as a notch, groove, or the like, that is configured to receive and retain a reciprocal protuberance 1406 extending from the IMD 1404. A wire 1408 retained within the connection tool 1402 is threadably secured (either by itself, or through a stylet) to a proximal end 1410 of the IMD 1404. The wire 1408 is pulled back into the connection tool 1402, thereby moving the IMD 1404 into the connection tool 1402. The protuberance 1406 is retained by the securing feature 1405 so that the IMD 1404 is prevented from rotating with respect to the connection tool 1404. As long as the wire 1408 continues to pull back on the IMD 1404, the IMD 1404 remains secured to the connection tool 1400. In order to disconnect the IMD 1404 from the connection tool 1402, the wire 1408 is rotated in a disconnecting direction, such as through the graspable device-connection control handle 126 shown in FIG. 1. Once the wire 1408 is disconnected from the IMD 1404, the device-engaging member 1400 may disconnect from the IMD 1410.

Alternatively, the IMD 1404 may include the securing feature 1404, while the device-engaging member 1400 may include the reciprocal protuberance 1406.

FIG. 15 illustrates a connection tool 1500, according to an embodiment. In this embodiment, the connection tool 1500 includes a pull wire 1502 within a deflectable member 1504. A distal end 1502 of the pull wire 1502 is connected to a device-engaging member 1507, having a joint 1508 that connects opposed securing members 1510, such as prongs, beams, clasps, an expandable ring, or the like. The spring tension of the securing members 1510 causes the opposed spring members 1510 to open away from one another in the at-rest position, as shown in FIG. 15.

The connection tool 1500 may be used in conjunction with the IMD 1104 shown in FIG. 11. In order to secure the device-engaging member 1507 to the IMD, the securing members 1510 are positioned around a protuberance of the IMD. An operator then pulls back the wire 1502, such as with the graspable device-connection control handle 126 shown in FIG. 1. As the securing members 1510 retreat into the deflectable member 1504, the inner diameter of the deflectable member 1504 squeezes the securing members 1510 together, and around the protuberance of the IMD, thereby securing the IMD to the device-engaging member 1507. Inner surfaces of the securing members 1510 may also include gripping members 1512, such as rubber pads, configured to securely grip the protuberance of the IMD.

In order to disconnect the IMD from the device-engaging member 1507, the wire 1502 is pushed outwardly so that the securing members 1510 pass out of the deflectable member 1504. The natural spring force of the spring members 1510 causes them to open up and release the protuberance from their grip.

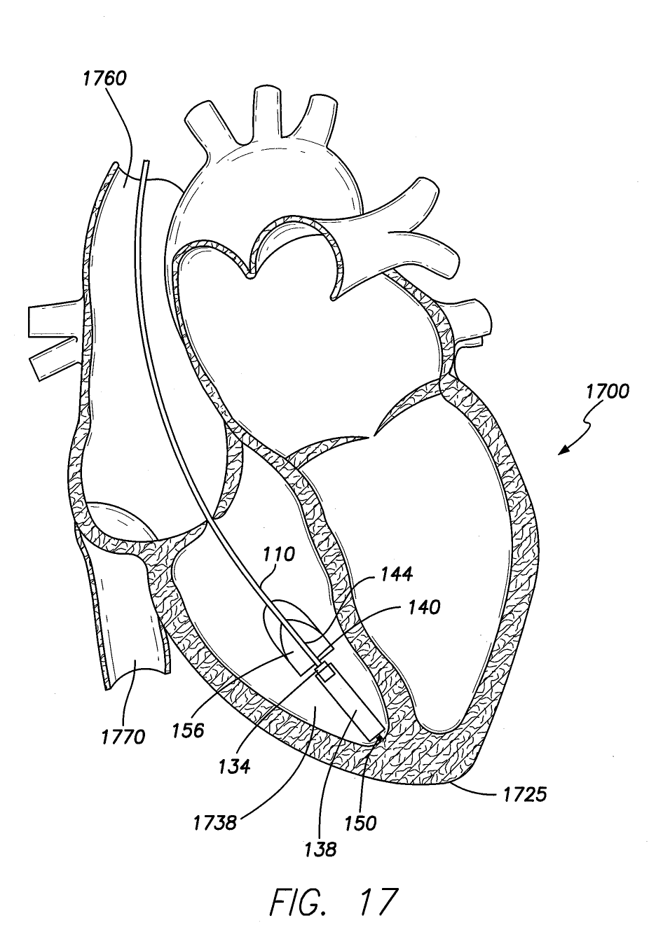

FIG. 16 illustrates a device-securing member 1600 aligned with a securing member 1602 extending from an IMD 1604, according to an embodiment. The device-securing member 1600 may be a key, such as a tab, ridge, post, hemispherical protrusion, or other such protuberance extending from a wire 1606, which may connect to the graspable device-connection control handle 126 (shown in FIG. 1). Optionally, the device securing member 1600 may extend from a deflectable member, such as the deflectable member 144 shown in FIGS. 3 and 4.

In order to connect the device-securing member 1600 to the IMD 1604, the securing member 1602 is aligned with an open end 1608 of a channel 1610 formed through a wall 1612 of the securing member 1602. The channel 1610 extends from the open end 1608 to a linear insertion portion 1614. The linear insertion portion 1614, in turn, connects to an expanded retaining portion 1618, which may be perpendicular to the linear insertion portion 1614.

The wire 1606 fits within a central passage 1620 of the securing member 1602. The wire 1606 is pushed into the central passage 1620 such that the device-securing member 1600 passes into the linear insertion portion 1614. Once the device-securing member 1600 abuts against an edge of the wall 1612 defining a distal portion of the retaining portion 1618, the wire 1606 is rotated in a securing or connecting direction so that the device-securing member 1600 moves out of the insertion portion 1614 and into a terminal end 1622 of the retaining portion 1618. As such, the device-securing member 1600 is no longer aligned with the insertion portion 1614, and cannot pass therein. Therefore, the device-securing member 1600 is secured to the IMD 1604.

In order to remove the device-securing member 1600 from the IMD 1604, the wire 1606 is rotated so that the device-securing member 1600 is aligned with the insertion portion 1614. The device-securing member 1600 may then be removed from the securing member 1602 by way of the wire 1606 pulling out of the central passage 1620.

As noted above, the device-securing member 1600 may extend from the deflectable member 144 or other portions of the connection tool 140 (shown in FIG. 2, for example). In this embodiment, the central passage 1620 of the securing member 1602 is sized and configured to receive the deflectable member 144, for example.

Additionally, alternatively, the IMD 1604 may include a key, tab, slot, or the like, while the connection tool and/or deflectable member may include channel 1610, for example.

Referring again to FIGS. 1-6, in order to implant the IMD 138, for example, into tissue of a patient, the IMD 138 is first secured to the device-engaging member 134. The graspable device-connection control handle 126 is used to maneuver the device-engaging member 134 into a secure relationship with the IMD 138, such as discussed above with respect to FIGS. 7-16. The operator then pulls back the IMD 138 into the protective covering member 156.

The operator then manipulates the main handle assembly 102 to navigate the IMD 138 to an implantation site. For example, the operator may grasp the fixed portion 112 of the main handle assembly 102 and rotate the rotatable portion 114, which deflects the deflectable member 144 as discussed above with respect to FIG. 3. The drip line 118 may be used to flush internal channels of the introducer 110 and the connection tool 140.

FIG. 17 illustrates the IMD 138 being implanted within a heart 1700 of a patient, according to an embodiment. The IMD 138 may be implanted entirely within the heart 1700. The IMD 138 may be secured to a wall defining the right ventricle 1738 proximate an apex 1725 of the heart 1700.

While the heart 1700 is shown, the implantation system 100 (shown in FIGS. 1 and 17) may be used to introduce the IMD 138 into various other organs, tissues, vasculature, such as arteries and veins, and the like, of the patient.

Referring to FIGS. 1-6 and 17, the main handle assembly 102 is operated to navigate the IMD 138 into the right ventricle 1738 through the superior vena cava 1760, for example. Optionally, the IMD 138 may be navigated into the right ventricle 1738 through the inferior vena cava 1770, for example. Further, the IMD 138 may be navigated into other chambers of the heart 1700. Additionally, the IMD 138 may be implanted into various other tissues within the patient. The heart 1700 shown in FIG. 17 is merely exemplary.

Once the IMD 138 is proximate the implantation site, the operator engages the graspable handle 136 and/or the main handle assembly 102 to push the IMD 138 out of the protective covering member 156 so that the securing helix 150 is urged into tissue, such as a wall portion defining the right ventricle 1738. The IMD 138 is then rotated to screw the securing helix 150 into the wall portion. Once the IMD 138 is secured to the wall portion, the wire 125 within the introducer 110 is then rotated in the disconnecting direction to disengage the device-engaging member 134 from the IMD 138. Once the device-engaging member 134 disengages the IMD 138, the device-engaging member 134, the connection tool 140, and the introducer 110 may be removed from the IMD 138 and removed from the heart 1700, leaving the disconnected IMD 138 implanted to the heart wall within the right ventricle 1738.

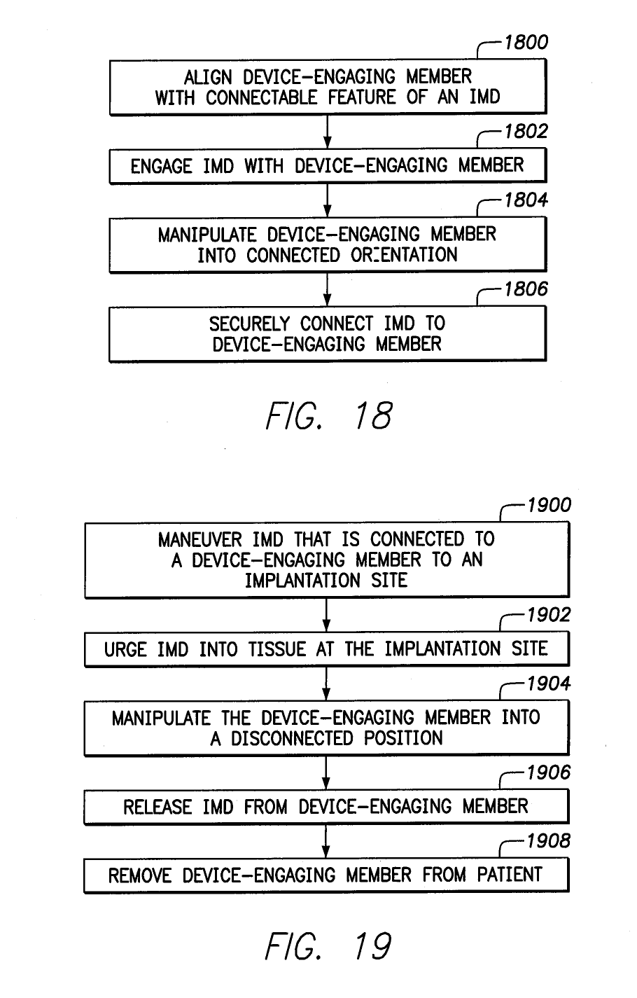

FIG. 18 illustrates a flow chart of a method of securing an implantable medical device to a device-engaging member. At 1800, the device-engaging member is aligned with a connectable feature of an IMD. The connectable feature may be a recessed cavity, a protuberance, or the like, such as shown and described with respect to FIGS. 1-17.

Next, at 1802, the device-engaging member engages the IMD. For example, the spreadable connectors of the device-engaging member are moved into a recessed cavity of the IMD.

Next, at 1804, the device-engaging member is manipulated into a connected orientation. For example, a wire connected to the device-engaging member may be rotated, pulled, or pushed into a securing or connection direction, thereby transitioning the device-engaging member into the connected position. At 1805, the IMD is securely connected to the device-engaging member when the device-engaging member mates with the IMD in the connected orientation.

FIG. 19 illustrates a flow chart of a method of implanting an IMD into a tissue within a patient, according to an embodiment. At 1900, an implantation system is used to maneuver an IMD that is connected to a device-engaging member to an implantation site within a patient, such as within a heart of the patient.

Next, at 1902, the IMD is urged into tissue at the implantation site. For example, a connection tool may be operated to push the IMD out of a protective covering member of the connection tool so that a helix of the IMD is urged into the tissue. The connection tool may then be rotated in order to securely anchor the IMD, via the helix, into the tissue of the patient.

After the IMD is securely anchored into the tissue of the patient, the device-engaging member is manipulated into a disconnected position. For example, a stylet of the device-engaging member may be rotated to withdraw from between opposed connectors. The IMD is related from the device-engaging member at 1906. Once in the disconnected position or orientation, the device-engaging member is removed from the IMD at 1908, leaving the IMD anchored into the tissue of the patient.

Thus, embodiments provide a system and method of implanting an IMD within a patient. Embodiments provide a system and method for quickly and easily connecting and disconnecting an IMD from a device-engaging member of an implantation system.

Moreover, embodiments provide a device-engaging member that gently, smoothly, and easily releases from the IMD. Thus, embodiments provide an implantation system that is less susceptible to pulling and tugging the IMD (and therefore patient tissue) when releasing the device-engaging member from the IMD.

Embodiments provide a system and method for connecting and disconnecting a device-engaging member to and from an IMD through a single, simple movement of a device-connection control handle. For example, the single movement may be a rotation of the device-connection control handle. Alternatively, the single movement may be a pulling or pushing movement of the device-connection control handle.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A system for implanting an implantable medical device (IMD) within a patient, the system comprising:
a main handle assembly having proximal and distal ends;
a device-connection control handle connected to the proximal end of the main handle assembly;
an introducer connected to the distal end of the main handle assembly; and
a connection tool extending from the introducer, wherein the connection tool comprises a device-engaging member configured to change at least one of shape or orientation to selectively connect to and disconnect from the IMD, wherein the device-connection control handle is operatively connected to the device-engaging member and the device-connection control handle is configured to manipulate the device-engaging member between connected and disconnected states by changing the at least one of the shape or orientation.

2. The system of claim 1, wherein the device-engaging member includes connectors that radially expand and contract outward and inward with respect to a longitudinal axis of the connection tool, thereby changing the shape of the device-engaging member.

3. The system of claim 1, wherein the device-engaging member includes connectors that move laterally inward and outward to change the shape between the disconnected and connected states.

4. The system of claim 1, wherein the connection tool comprises:
a base;
opposed spreadable connectors moveably secured to the base; and
a stylet configured to move between the opposed spreadable connectors in order to spread the opposed spreadable connectors into the connected state, and recede away from the opposed spreadable connectors so that the opposed spreadable connectors retract toward one another into the disconnected state.

5. The system of claim 4, wherein the IMD comprises either a recessed cavity or a protuberance, and wherein the opposed spreadable connectors are configured to engage the recessed cavity or the protuberance in the connected state.

6. The system of claim 4, wherein the opposed spreadable connectors are configured to pivot with respect to the base.

7. The system of claim 4, wherein the opposed spreadable connectors are configured to linearly move with respect to the base.

8. The system of claim 1, wherein the connection tool comprises a deflectable member.

9. The system of claim 8, wherein the deflectable member comprises an elongated braid having a metal mesh.

10. The system of claim 1, wherein the connection tool comprises a protective covering member configured to protect the IMD during navigation to an implantation site, wherein the IMD is moved out of the protective covering member at the implantation site.

11. The system of claim 1, wherein the device-engaging member comprises:
a securing cuff operatively connected to the device-connection control handle; and
at least one securing member configured to engage the IMD, wherein the securing cuff is configured to engage the at least one securing member.

12. The system of claim 11, wherein the securing cuff is connected to a coupling member that is operatively connected to the device-connection control handle.

13. The system of claim 12, wherein the securing cuff is connected to the coupling member through at least one beam.

14. The system of claim 12, wherein the securing cuff and the coupling member magnetically attract or repel one another.

15. The system of claim 1, wherein one of the device-engaging member or the IMD comprises a distal block configured to pass into a securing channel formed in the other of the device-engaging member or the IMD.

16. The system of claim 1, wherein the device-engaging member comprises one of a securing feature or a protuberance, and the IMD comprises the other of the securing feature or the protuberance, and wherein the securing feature receives and retains the protuberance when the IMD is pulled into the device-engaging member.

17. The system of claim 1, wherein the device-engaging member comprises securing members pivotally connected to one another, and wherein the device-engaging member is configured to be pushed out of and pulled into a deflectable member through operation of the device-connection control handle.

18. The system of claim 1, wherein one of the device-engaging member or the IMD comprises a key, and the other of the device-engaging member or the IMD comprises a channel configured to receive the key, wherein the channel comprises an insertion portion connected to a retaining portion, and wherein the device-engaging member is maneuvered into the retaining portion by the device-connection control handle in the connected orientation.

19. A method of connecting an implantable medical device (IMD) to a device-engaging member extending from a connection tool of an implantation system, the method comprising:
aligning the device-engaging member with a connecting interface of the IMD;
moving the device-engaging member into or onto the connecting interface;
manipulating a device-connection control handle of the implantation system;
changing at least one of shape or orientation of the device-engaging member, by the manipulating, from a disconnected state to a connected state while the device-engaging member is on or within the connecting interface; and
connecting the IMD to the device-engaging member through the changing at least one of shape or orientation.

20. The method of claim 19, wherein the changing at least one of shape or orientation comprises radially expanding or contracting connectors of the device-engaging member outward or inward with respect to a longitudinal axis of the connection tool.

21. The method of claim 19, wherein the changing at least one of shape or orientation comprises laterally moving connectors of the device device-engaging member inward or outward.

22. The method of claim 19, wherein the manipulating comprises rotating the device-connection control handle.

23. The method of claim 19, wherein the manipulating comprises pushing or pulling the device-connection control handle.

24. A method of implanting an implantable medical device (IMD) within a patient, the method comprising:
connecting the IMD to a device-engaging member at a distal end of a connection tool of an implantation system;
maneuvering the IMD to an implantation site through the implantation system;
advancing the IMD out of a protective covering member of the connection sheath into the implantation site;
manipulating the implantation system to securely anchor the IMD into tissue at the implantation site;
manipulating a device-connection control handle of the implantation system;
changing at least one of shape or orientation of the device-engaging member by the manipulating the device-connection control handle; and
disconnecting the device-engaging member from the IMD through the changing at least one of shape or orientation of the device-engaging member.

25. The method of claim 24, wherein the changing at least one of shape or orientation comprises radially expanding or contracting connectors of the device-engaging member outward or inward with respect to a longitudinal axis of the connection tool.

26. The method of claim 24, wherein the changing at least one of shape or orientation comprises laterally moving connectors of the device device-engaging member inward or outward.

27. The method of claim 24, wherein the manipulating the device-connection control handle comprises rotating the device-connection control handle.

28. The method of claim 24, wherein the manipulating the device-connection control handle comprises pushing or pulling the device-connection control handle.
